(19) United States
(12) Patent Application Publication
(10) Pub. No.: US 2026/0309635 A1
(43) Pub. Date: Oct. 8, 2026

(54) OPTICAL INSPECTION DEVICE, OPTICAL INSPECTION SYSTEM, AND OPTICAL INSPECTION METHOD

(71) Applicant: Samsung Display Co., Ltd., Yongin-si (KR)

(72) Inventor: Hyungjin Lee, Yongin-si (KR)

(21) Appl. No.: 19/572,610

(22) Filed: Mar. 19, 2026

(30) Foreign Application Priority Data
Apr. 3, 2025 (KR) 10-2025-0043609

(51) Int. Cl. G01N 21/84; G01N 21/01; G01N 33/569

(52) U.S. Cl. CPC G01N 21/84; G01N 21/01; G01N 33/56983; G01N 2201/0683

(57) ABSTRACT

Provided is an optical inspection device including a light source, at least one polarizer provided on a path of light emitted from the light source and including a polarization structure, and a sensor that detects light from the at least one polarizer, wherein the polarization structure of the at least one polarizer includes a connection structure that includes a plurality of microorganisms.

### CROSS-REFERENCE TO RELATED APPLICATION

The present application claims priority to and the benefit of Korean Patent Application No. 10-2025-0043609, filed on Apr. 3, 2025, in the Korean Intellectual Property Office, the entire content of which is hereby incorporated by reference.

### BACKGROUND

### 1. Field

One or more embodiments of the present disclosure relate to an optical inspection device. One or more embodiments also relate to an optical inspection system including the optical inspection device. One or more embodiments also relate to an optical inspection method using the optical inspection device or the optical inspection system.

### 2. Description of the Related Art

An optical inspection device may be used to identify characteristics of an object for inspection. For example, an optical inspection device may be used to determine whether a set or specific layer is provided in a set or certain area of a display panel which is an object for inspection. Light may be irradiated from a light source to the object for inspection (for example, the display panel), and light reflected from the object for inspection (for example, the display panel) may be detected by a sensor. The light reflected from the object for inspection (for example, the display panel) may be polarized by one or more polarizers before entering the sensor. Design factors such as, for example, the structure, polarization angles, and number of polarizers may suitably vary depending on the type or kind of layer for which the arrangement is to be determined.

### SUMMARY

Polarization structures of polarizers may have dimensions on a nanometer scale, and artificially forming and integrating such polarization structures may present difficulties.

Additional aspects of embodiments will be set forth in part in the description which follows and, in part, will be apparent from the description, or may be learned by practice of the presented embodiments of the disclosure.

According to one or more embodiments, an optical inspection device includes a light source, at least one polarizer provided on a path of light emitted from the light source and including a polarization structure, and a sensor that detects light from the at least one polarizer, wherein the polarization structure of the at least one polarizer includes a connection structure that includes a plurality of microorganisms.

In an embodiment, the connection structure that includes the plurality of microorganisms may be provided as a mesh structure that includes holes that generally extend in one direction.

In an embodiment, the plurality of microorganisms may include filamentous bacteriophages.

In an embodiment, the at least one polarizer may include a film that transmits the light emitted from the light source.

In an embodiment, the at least one polarizer may include a mirror that reflects the light emitted from the light source.

In an embodiment, the optical inspection device may further include a processor connected to the sensor and that includes an algorithm that creates an image based on the light detected by the sensor.

In an embodiment, the at least one polarizer may include a protective film that covers the polarization structure.

In an embodiment, the at least one polarizer may include a plurality of polarizers having different polarization angles and provided continuously on the path of the light emitted from the light source.

According to one or more embodiments, an optical inspection system includes a light source, an object for inspection provided on a path of light emitted from the light source, at least one polarizer provided on the path of the light emitted from the light source and reflected from the object for inspection, and that includes a polarization structure, and a sensor that detects light from the at least one polarizer, wherein the polarization structure of the at least one polarizer includes a connection structure that includes a plurality of microorganisms.

In an embodiment, the connection structure that includes the plurality of microorganisms may be provided as a mesh structure that includes holes that generally extend in one direction.

According to one or more embodiments, an optical inspection method includes irradiating light from a light source toward an object for inspection, polarizing, at least once, light reflected from the object for inspection, using at least one polarizer, and detecting, using a sensor, the light polarized by the at least one polarizer, wherein a polarization structure of the at least one polarizer includes a connection structure that includes a plurality of microorganisms.

In an embodiment, the connection structure that includes the plurality of microorganisms may be provided as a mesh structure that includes holes that generally extend in one direction.

In an embodiment, the plurality of microorganisms may include filamentous bacteriophages.

In an embodiment, the at least one polarizer may include a film that transmits light, and in the polarizing of the light, the light may be polarized by the polarization structure and may be transmitted through the film.

In an embodiment, the at least one polarizer may include a mirror that reflects light, and in the polarizing of the light, the light may be polarized by the polarization structure and may be reflected by the mirror.

In an embodiment, the method may further include forming an image of the object for inspection, based on the light detected by the sensor.

In an embodiment, the at least one polarizer may include a protective film covering the polarization structure, and the forming of the image may include compensating for an effect of the protective film on the light.

In an embodiment, in the polarizing of the light, the light reflected from the object for inspection may be polarized a plurality of times using a plurality of polarizers provided continuously on a path of light and having different polarization angles.

In an embodiment, the object for inspection may include a display panel, and characteristics of a set or specific area of the display panel may be identified based on the light detected using the sensor.

In an embodiment, the object for inspection may be moved to identify whether a set or specific layer of the display panel is present and to identify a boundary of the set or specific layer.

### BRIEF DESCRIPTION OF THE DRAWINGS

The above and other aspects and features of certain embodiments of the disclosure will be more apparent from the following description taken in conjunction with the accompanying drawings, in which:

FIG. 1 is a schematic view of an optical inspection system according to an embodiment;

FIG. 2 is a schematic view of an optical inspection system according to an embodiment;

FIGS. 3A and 3B are enlarged views of a polarization structure according to an embodiment;

FIG. 3C is a schematic view of a portion of the polarization structure of FIGS. 3A and 3B;

FIG. 4A and 4B are enlarged views of a polarization structure according to an embodiment;

FIG. 4C is a schematic view of a portion of the polarization structure of FIGS. 4A and 4B;

FIG. 5 is a schematic plan view of a display panel according to an embodiment;

FIG. 6 is an equivalent circuit diagram of a pixel of a display panel according to an embodiment;

FIG. 7 is a cross-sectional view of a display area of a display panel according to an embodiment;

FIG. 8A is a graph of a transmittance ratio with respect to a polarization angle according to an embodiment;

FIG. 8B is a graph of a transmittance ratio with respect to a polarization angle according to an embodiment;

FIG. 9A is a graph of a reflectance ratio with respect to a polarization angle according to an embodiment;

FIG. 9B is a graph of a reflectance ratio with respect to a polarization angle according to an embodiment;

FIGS. 10A, 10B, 10C, and 10D are images of set or certain areas of an object for inspection;

FIG. 11A is a plan view of a portion of a display panel according to an embodiment; and

FIG. 11B is a plan view of a portion of a display panel according to an embodiment.

### DETAILED DESCRIPTION

Reference will now be made in more detail to embodiments, examples of which are illustrated in the accompanying drawings, wherein like reference numerals refer to like elements throughout the specification. In this regard, the present embodiments may have different forms and should not be construed as being limited to the descriptions set forth herein. Accordingly, the embodiments are merely described below, by referring to the figures, to explain aspects of the present description. As used herein, the term "and/or" includes any and all combinations of one or more of the associated listed items. Throughout the disclosure, the expression "at least one of a, b or c" indicates only a, only b, only c, both a and b, both a and c, both b and c, all of a, b, and c, or variations thereof.

Various suitable modifications may be applied to the present embodiments, and example embodiments will be illustrated in the drawings and described in the detailed description section. The effect and features of embodiments of the disclosure, and a method to achieve the same, will be clearer referring to the detailed descriptions below together with the drawings. The disclosed embodiments may be implemented in various suitable forms, not by being limited to the embodiments presented below.

Hereinafter, embodiments of the present disclosure will be described in more detail with reference to the accompanying drawings, and in the description with reference to the drawings, the same or corresponding components are indicated by the same reference numerals and redundant descriptions thereof are omitted.

In the present specification, it will be understood that although the terms "first," "second," and/or the like may be used herein to describe various components, these components should not be limited by these terms. These terms are only used to distinguish one component from another.

In the present specification, expressions in a singular form in the present specification may include expressions in a form of a plurality unless clearly specified otherwise in context.

In the present specification, it will be further understood that the terms “include,” “includes,” “including,” “comprise,” "comprises" and/or "comprising" used herein specify the presence of stated features or components, but do not preclude the presence or addition of one or more other features or components.

In the present specification, it will be understood that if (e.g., when) a layer, area, or component is referred to as being “on” or "formed on" another layer, area, or component, it can be directly or indirectly on or formed on the other layer, area, or component. For example, intervening layers, areas, or components may be present.

Sizes of components in the drawings may be exaggerated or reduced for convenience of explanation. For example, because sizes and thicknesses of components in the drawings may be arbitrarily illustrated for convenience of explanation, the following embodiments are not limited thereto.

If (e.g., when) a certain embodiment may be implemented differently, a specific process order may be performed differently from the described order. For example, two consecutively described processes may be performed substantially at the same time or performed in an order opposite to the described order.

In the present specification, the expression "A and/or B" represents A, B, or A and B. In the present specification, the expression "at least one of A or B" represents A, B, or A and B.

In the present specification, it will be understood that if (e.g., when) a layer, area, or component is referred to as being (for example, electrically) connected to another layer, area, or component, the layers, areas, or components may be directly (for example, electrically) connected to each other, and/or may be indirectly (for example, electrically) connected to each other via another layer, area, or component therebetween.

The x-axis, the y-axis, and the z-axis are not limited to three axes of the rectangular coordinate system, and may be interpreted in a broader sense. For example, the x-axis, the y-axis, and the z-axis may be perpendicular to one another, or may not be perpendicular to one another.

Terms such as “substantially,” “about,” and “approximately” are used as relative terms and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or ± 30%, 20%, 10%, 5% of the stated value.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6. Applicant therefore reserves the right to amend this specification, including the claims, to expressly recite any sub-range subsumed within the ranges expressly recited herein.

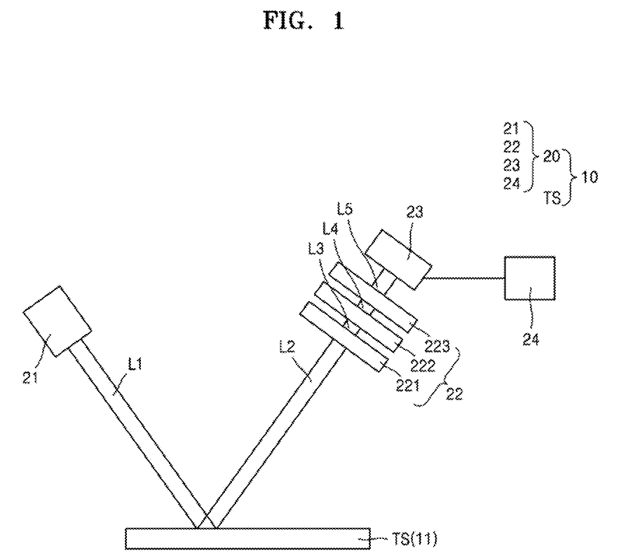

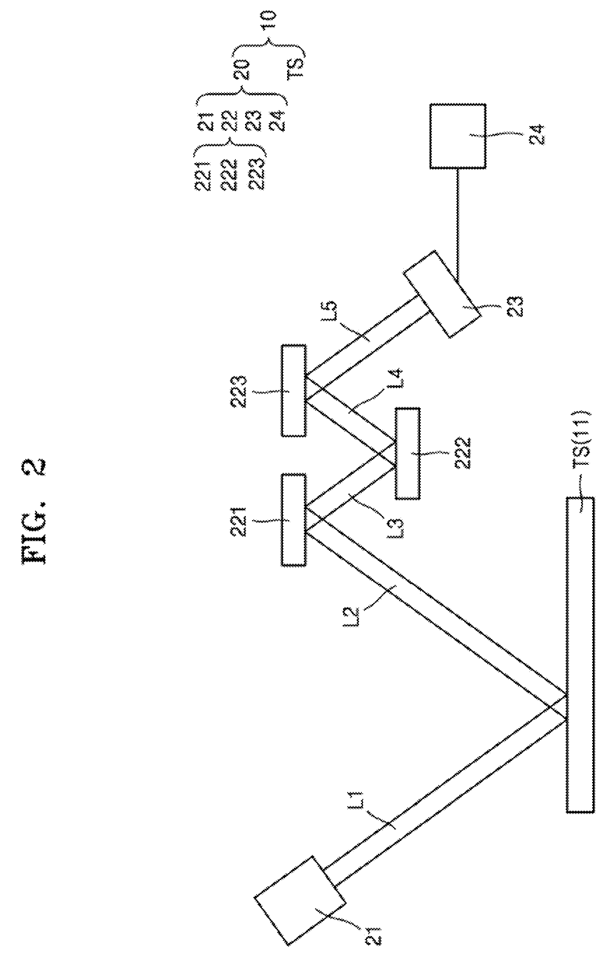

FIG. 1 is a schematic view of an optical inspection system according to an embodiment. FIG. 2 is a schematic view of an optical inspection system according to an embodiment. FIG. 1 shows an embodiment of an optical inspection system in a transmission-type configuration, and FIG. 2 shows an embodiment of an optical inspection system in a reflection-type configuration.

Referring to FIG. 1, an optical inspection system 10 may include an optical inspection device 20 and an object TS for inspection. The object TS for inspection may be a target to be inspected by the optical inspection device 20. In an embodiment, the object TS for inspection may be a display panel 11. The optical inspection device 20 may include a light source 21, at least one polarizer 22, a sensor 23, and a processor 24.

The light source 21 may be configured to emit light, for example, a first light L1, in one direction, for example, toward the object TS for inspection. In an embodiment, the first light L1 may be a laser beam. In an embodiment, the object TS for inspection may be provided on a path of the first light L1. In an embodiment, the first light L1 may be light incident on the object TS for inspection, and a second light L2 may be light reflected from the object TS for inspection. In an embodiment, characteristics of the second light L2 may suitably vary depending on characteristics (for example, the presence or absence of a set or specific layer of the display panel 11) of the object TS for inspection, at a position on the object TS for inspection where reflection occurs. Therefore, in an embodiment, the characteristics of the second light L2 may differ from characteristics of the first light L1.

The second light L2 reflected from the object TS for inspection may be directed toward the at least one polarizer 22. In an embodiment, the at least one polarizer 22 may include a plurality of polarizers 22. In an embodiment, the at least one polarizer 22 may include a first polarizer 221, a second polarizer 222, and a third polarizer 223. In an embodiment, the first polarizer 221, the second polarizer 222, and the third polarizer 223 may be provided sequentially or continuously on a path of light, for example, a path of the second light L2.

In an embodiment, the second light L2 may be light incident on the first polarizer 221, and a third light L3 may be light transmitted from the first polarizer 221. In an embodiment, the third light L3 may be light incident on the second polarizer 222, and a fourth light L4 may be light transmitted from the second polarizer 222. In an embodiment, the fourth light L4 may be light incident on the third polarizer 223, and a fifth light L5 may be light transmitted from the third polarizer 223. However, the disclosure is not necessarily limited to a set or specific number of polarizers, and the number of polarizers may suitably vary.

In an embodiment, the first polarizer 221, the second polarizer 222, and the third polarizer 223 may have different polarization angles. In an embodiment, the polarization angle of the first polarizer 221 may be 0 degrees, the polarization angle of the second polarizer 222 may be 45 degrees, and the polarization angle of the third polarizer 223 may be 90 degrees. However, the disclosure is not necessarily limited to a set or specific polarization angle, and the polarization angle of each of the polarizers may suitably vary.

In an embodiment, the at least one polarizer 22 may include a polarization structure to polarize incident light. In an embodiment, as further described herein, the polarization structure of the at least one polarizer 22 may include a connection structure of microorganisms. In an embodiment, the polarization structure of the at least one polarizer 22 may include a connection structure formed by filterable microorganisms, for example, viruses. In an embodiment, the polarization structure of the at least one polarizer 22 may include a connection structure formed by bacteriophages. In an embodiment, the bacteriophages may be filamentous bacteriophages.

In an embodiment, the at least one polarizer 22 may include a polarization structure and may include a film that transmits light. In an embodiment, the polarization structure may be provided on one surface of the film that faces incident light and/or on one surface of the film from which transmitted light emerges, and/or may be embedded within the film. For example, the first polarizer 221 may include a film and a polarization structure that polarizes the second light L2, wherein the polarization structure may be provided on one surface of the film facing the second light L2 and/or on one surface of the film from which the third light L3 emerges, and/or may be embedded within the film.

In an embodiment, the at least one polarizer 22 may further include a protective film that protects the polarization structure. In an embodiment, the protective film may be provided to cover the polarization structure. In an embodiment, the protective film may be formed by dipping the polarization structure (and the film) into a resin and/or the like. As described above, because the polarization structure of the at least one polarizer 22 may include a connection structure of microorganisms, the polarization structure may be vulnerable to external air and/or impacts. Destruction and/or deformation of the connection structure of the microorganisms may lead to destruction and/or deformation of the polarization structure, which may in turn cause malfunction of the at least one polarizer 22, and further, the optical inspection device 20. The protective film may cover the polarization structure to protect the polarization structure and ensure or improve the performance of the at least one polarizer 22.

The sensor 23 may receive and/or detect light that has passed through the at least one polarizer 22. In an embodiment, the sensor 23 may detect the fifth light L5 from the at least one polarizer 22, for example, the third polarizer 223. In an embodiment, the sensor 23 may include a light detection sensor.

The processor 24 may be connected to the sensor 23. In an embodiment, the processor 24 may be configured to form an image of the object TS for inspection, based on the light detected by the sensor 23. In an embodiment, the processor 24 may include an algorithm that forms (and/or creates) an image based on the light detected by the sensor 23. In an embodiment, the algorithm of the processor 24 may be adjusted according to factors such as the number of polarizers 22, the type (or kind) of polarization structure, the polarization angle, the number of polarization angles, and the type (or kind) of object TS for inspection. In an embodiment, the algorithm of the processor 24 may be adjusted considering light distortion caused by the protective film of the at least one polarizer 22.

Referring to FIG. 2, except for some characteristics of the at least one polarizer 22, the remaining characteristics are the same as the characteristics of the embodiment described with reference to FIG. 1, and thus repeated descriptions thereof may not be repeated here. In the embodiment shown in FIG. 2, the at least one polarizer 22, for example, the first polarizer 221, the second polarizer 222, and the third polarizer 223, may be provided in a reflection-type configuration.

In an embodiment, the second light L2 may be light incident on the first polarizer 221, and the third light L3 may be light reflected from the first polarizer 221. In an embodiment, the third light L3 may be light incident on the second polarizer 222, and the fourth light L4 may be light reflected from the second polarizer 222. In an embodiment, the fourth light L4 may be light incident on the third polarizer 223, and the fifth light L5 may be light reflected from the third polarizer 223.

In an embodiment, the at least one polarizer 22 may include a polarization structure and may include a mirror that reflects light. In an embodiment, the polarization structure may be provided on one surface of the mirror facing incident light. In an embodiment, the first polarizer 221 may include a mirror and a polarization structure that polarizes the second light L2, and the polarization structure may be provided on one surface of the mirror facing the second light L2. In an embodiment, the second polarizer 222 may include a mirror and a polarization structure that polarizes the third light L3, and the polarization structure may be provided on one surface of the mirror facing the third light L3. In an embodiment, the third polarizer 223 may include a mirror and a polarization structure that polarizes the fourth light L4, and the polarization structure may be provided on one surface of the mirror facing the fourth light L4.

In an embodiment, the sensor 23 may receive and/or detect light reflected from the at least one polarizer 22.

Hereinafter, an optical inspection method using the optical inspection system 10 is described.

In an embodiment, the optical inspection method may include irradiating light (for example, the first light L1) from the light source 21 toward the object TS for inspection, polarizing, at least once, light (for example, the second light L2) reflected from the object TS for inspection, using the at least one polarizer 22, and detecting, using the sensor 23, light (for example, the fifth light L5) polarized by the at least one polarizer 22. In an embodiment, in the polarizing of the light (for example, the second light L2), the light may be polarized three times using the plurality of polarizers 22, for example, the first polarizer 221, the second polarizer 222, and the third polarizer 223. However, the disclosure is not limited to polarizing light a set or specific number of times, and the number of polarizations may suitably vary.

In an embodiment, if (e.g., when) the optical inspection system 10 in a transmission-type configuration, as shown in FIG. 1, is used, in the polarizing of the light, the light may be polarized by the polarization structure provided in the at least one polarizer 22 and may pass through the film. In an embodiment, if (e.g., when) the optical inspection system 10 in a reflection-type configuration, as shown in FIG. 2, is used, in the polarizing of the light, the light may be polarized by the polarization structure provided in the at least one polarizer 22 and may be reflected from the mirror.

In an embodiment, the optical inspection method may further include forming an image of the object TS for inspection, based on the light detected by the sensor 23. In an embodiment, the forming of the image may be performed via the algorithm provided in the processor 24. In the embodiment in which the protective film covering the polarization structure is provided in the at least one polarizer 22, the forming of the image of the optical inspection method may include compensating for an effect of the protective film on the light.

In an embodiment, the object TS for inspection may include the display panel 11, and the optical inspection method may include identifying characteristics of a set or specific area of the display panel 11, based on the light detected using the sensor 23. In an embodiment, the optical inspection method may include determining whether a set or specific layer is provided in an area of the display panel 11 where the light has been irradiated, based on the light detected by the sensor 23. In an embodiment, the optical inspection method may include moving the object TS for inspection, for example, the display panel 11. In an embodiment, the optical inspection method may include identifying a boundary of the set or specific layer by moving the display panel 11 while continuously checking whether the set or specific layer of the display panel 11 is provided.

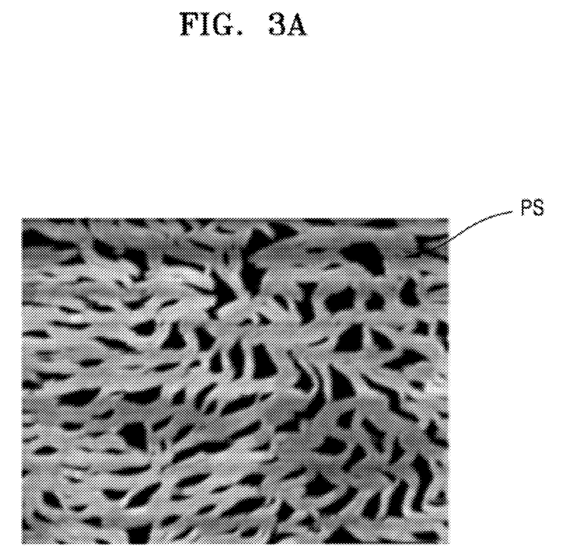

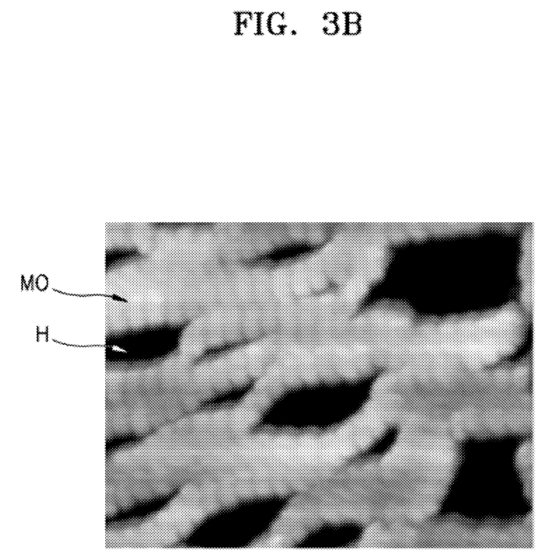

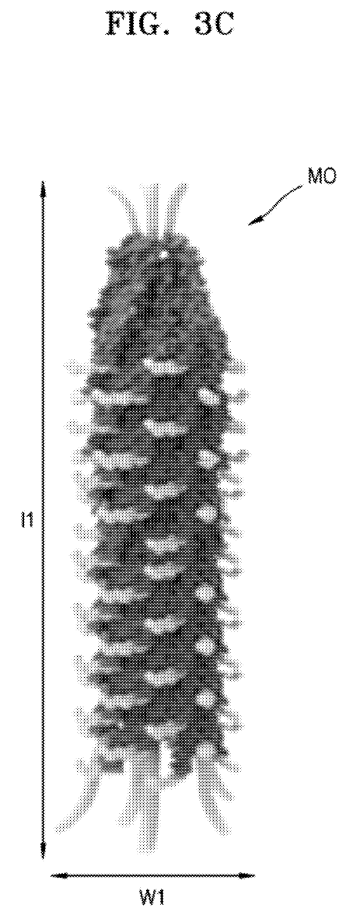

FIGS. 3A and 3B are enlarged views of a polarization structure according to an embodiment. FIG. 3B may be an enlarged view at a higher magnification than FIG. 3A. In embodiments, FIG. 3B may be a further enlarged view of a portion of FIG. 3A. FIG. 3C is a schematic view of a portion of the polarization structure of FIGS. 3A and 3B.

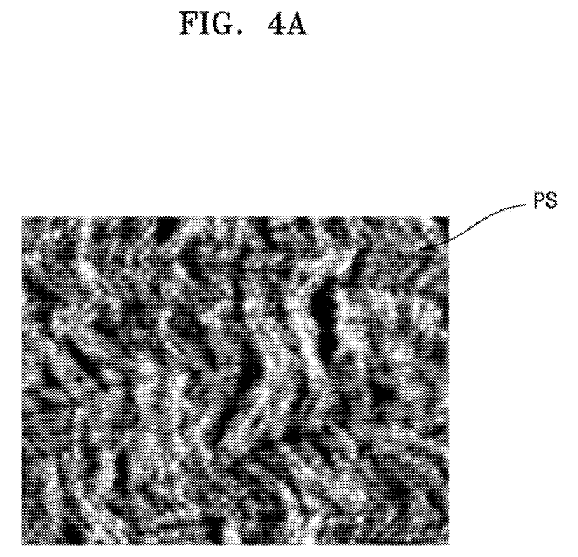

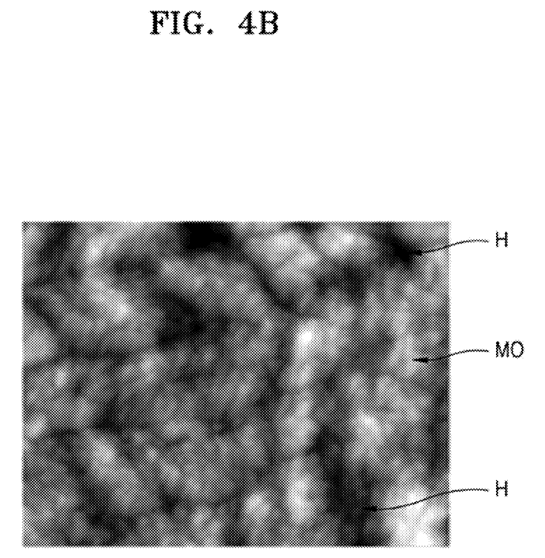

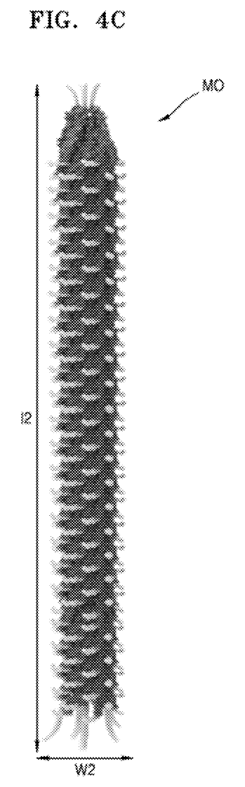

FIG. 4A and 4B are enlarged views of a polarization structure according to an embodiment. FIG. 4B may be an enlarged view at a higher magnification than FIG. 4A. In embodiments, FIG. 4B may be a further enlarged view of a portion of FIG. 4A. FIG. 4C is a schematic view of a portion of the polarization structure of FIGS. 4A and 4B.

Referring to FIGS. 3A, 3B, 4A, and 4B, a polarization structure PS may include a connection structure formed by a plurality of microorganisms MO. For example, the polarization structure PS may be formed by the connection structure of the polarization structure PS. In an embodiment, the microorganisms MO may include viruses. In an embodiment, the viruses may include bacteriophages. In an embodiment, the bacteriophages may include filamentous bacteriophages. In an embodiment, the bacteriophages may include M13 bacteriophages.

The polarization structure PS or the connection structure of the microorganisms MO may be provided as a mesh structure including holes H. In an embodiment, the connection structure may be formed such that the holes H generally have shapes that extend in one direction. For example, the holes H in the connection structure may generally have elongated or slender shapes that extend in one direction.

The microorganisms MO may prevent or reduce transmission of light, whereas the holes H in the connection structure may allow transmission of light. Because the connection structure may have a dimension on a nanometer (nm) scale and the holes H in the connection structure may have elongated shapes that extend in one direction, the connection structure of the microorganisms MO may be utilized to polarize light.

The connection structure of the microorganisms MO may be realized via self-replication and/or proliferation of the microorganisms MO. In an embodiment, an environment for self-replication of the microorganisms MO may be provided, and the microorganisms MO may be replicated for a set or certain period of time within the environment. In an embodiment, as shown in FIGS. 3C and 4C, the filamentous bacteriophages (for example, M13 bacteriophages) that form a filamentous structure may be used as the microorganisms MO.

The polarization structure PS for polarizing light may have a dimension on a nanometer (nm) scale, making artificial formation challenging. In embodiments, formation of a pattern of the connection structure utilized or required for the polarization structure PS relies on natural characteristics of the microorganisms MO, and the polarization structure may be easily formed by adjusting relatively controllable variables such as replication rate and time. Accordingly, time and cost required for forming the polarization structure PS may be reduced.

Comparing the embodiment (hereinafter, referred to as a first embodiment) shown in FIGS. 3A and 3B with the embodiment (hereinafter, referred to as a second embodiment) shown in FIGS. 4A and 4B, the holes H in the polarization structure PS in the second embodiment may have more elongated shapes. As further described herein, the degree of polarization of the polarization structure PS in the second embodiment may be greater than the degree of polarization of the polarization structure PS in the first embodiment.

FIGS. 3C and 4C show partial views of the polarization structure PS. The microorganisms MO may form a filamentous structure in which their ends (for example, front and rear ends) are connected to each other in an extended manner, and filaments may be connected to each other to form the polarization structure PS shown in FIGS. 3A, 3B, 4A, and 4B.

In an embodiment, a filament in the embodiment (first embodiment) shown in FIG. 3C may have a first width W1 and a first length l1, and a filament in the embodiment (second embodiment) shown in FIG. 4C may have the first width W1 and a second length l2. Because the ends of the microorganisms MO are connected to each other in an extended manner, the filaments in both the first and second embodiments may have the same first width W1. In an embodiment, the second length l2 may be greater than the first length l1. In an embodiment, the first length l1 may be about 240 nanometers (nm), and the second length l2 may be about 950 nanometers (nm). The length of a single filament may be adjusted by adjusting the time it takes for the microorganisms MO to self-replicate and/or proliferate. In an embodiment, the filament in the second embodiment may be formed via replication of the microorganisms MO over a longer period of time than the filament in the first embodiment.

The polarization structure PS in the first embodiment shown in FIGS. 3A and 3B may be formed via the filament in the first embodiment shown in FIG. 3C, and the polarization structure PS in the second embodiment shown in FIGS. 4A and 4B may be formed via the filament in the second embodiment shown in FIG. 4C. Because the filament in the second embodiment may have a greater length than the filament in the first embodiment, connection structures of the polarization structure PS in the second embodiment may have greater lengths than connection structures of the polarization structure PS in the first embodiment, and the holes H in the polarization structure PS in the second embodiment may have greater lengths than the holes H in the polarization structure PS in the first embodiment.

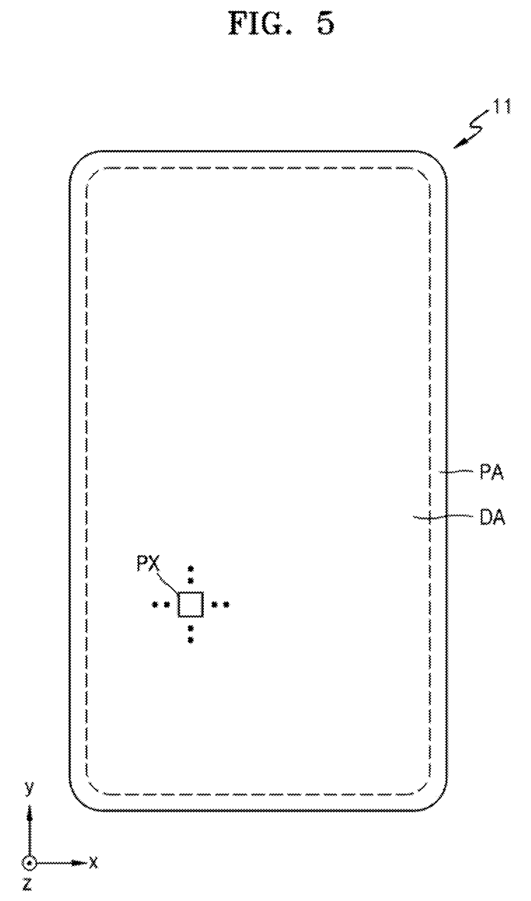

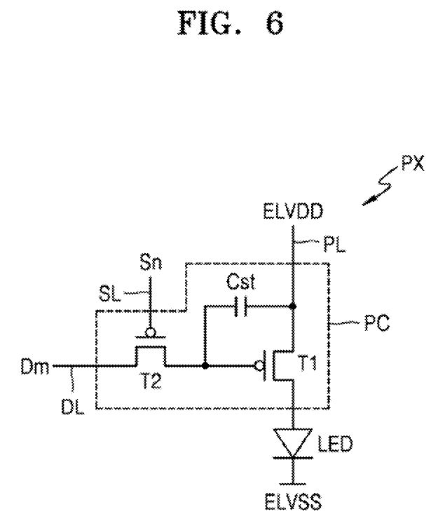

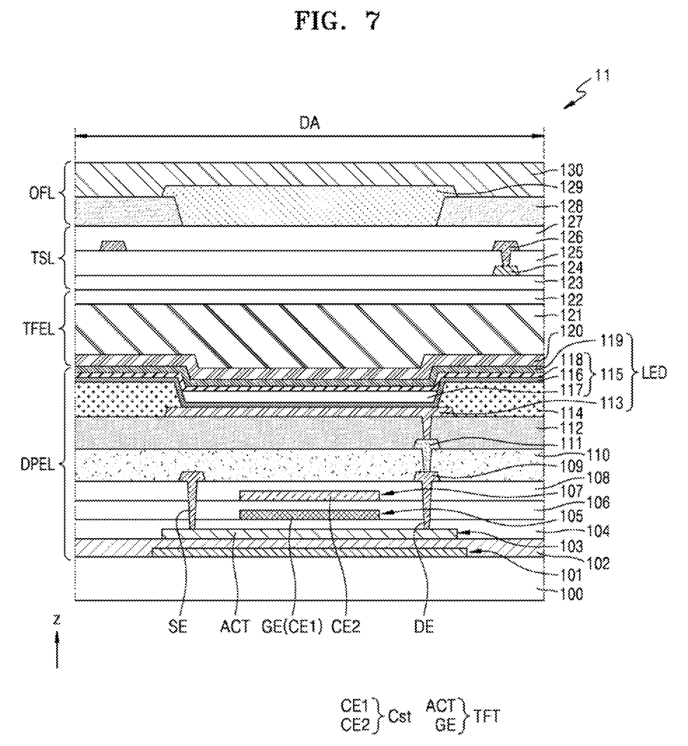

FIG. 5 is a schematic plan view of a display panel according to an embodiment. FIG. 6 is an equivalent circuit diagram of a pixel of a display panel according to an embodiment. FIG. 7 is a cross-sectional view of a display area of a display panel according to an embodiment.

Referring to FIG. 5, the display panel 11 may include a display area DA and a peripheral area PA outside the display area DA. For example, the display area DA and the peripheral area PA outside the display area DA may be defined in the display panel 11. In embodiments, the display panel 11 may include a substrate 100 (see FIG. 7), and the display area DA and the peripheral area PA may be defined on the substrate 100.

A pixel PX may be provided in the display area DA. The pixel PX may include at least one light-emitting diode LED and a pixel circuit PC configured to drive a light-emitting diode LED (see FIG. 6). A light-emitting diode driven by the pixel circuit PC may emit light of a set or specific color (e.g., a wavelength band). The display panel 11 may provide an image and/or video via light emitted from a plurality of light-emitting diodes provided in a plurality of pixels PX. In an embodiment, the pixel PX may include a group of sub-pixels. In an embodiment, one sub-pixel may be provided with one corresponding light-emitting diode and one corresponding sub-pixel circuit. In an embodiment, light-emitting diodes provided to each of the sub-pixels grouped into one pixel PX may emit light of different colors (e.g., wavelength bands). The peripheral area PA is a non-display area, and a signal line and/or a voltage line for driving a light-emitting diode may be provided in the peripheral area PA.

FIG. 5 shows that each of the display panel 11 and the display area DA has an approximately quadrangular shape having rounded corners, but the disclosure is not necessarily limited thereto. The display panel 11 and/or the display area DA may have various suitable shapes such as, for example, polygonal, circular, elliptical, and/or irregular shapes.

Referring to FIG. 6, the pixel circuit PC may be connected to the light-emitting diode LED to implement light emission of the pixels PX. In an embodiment, the light-emitting diode LED may be an organic light-emitting diode. The pixel circuit PC may include a first transistor T1, a second transistor T2, and a storage capacitor Cst. In an embodiment, the first transistor T1 may be a driving transistor, and the second transistor T2 may be a switching transistor. The second transistor T2 may be connected to a scan line SL and a data line DL, and may be configured to transmit, to the first transistor T1, a data signal Dm input via the data line DL according to a scan signal Sn input via the scan line SL.

The storage capacitor Cst is connected to the second transistor T2 and a driving voltage line PL, and is configured to store a voltage corresponding to a difference between a voltage received from the second transistor T2 and a driving voltage ELVDD supplied to the driving voltage line PL.

The first transistor T1 may be connected to the driving voltage line PL and the storage capacitor Cst, and may be configured to control a driving current flowing from the driving voltage line PL to the light-emitting diode LED according to a voltage value stored in the storage capacitor Cst. The light-emitting diode LED may emit light having a set or certain luminance according to a difference between a voltage provided by the first transistor T1 and a common voltage ELVSS.

However, the pixel circuit PC is not limited to the number and circuit design of thin-film transistors and capacitors described with reference to FIG. 6, and the number and circuit design of thin-film transistors and capacitors may be changed in various suitable ways.

Referring to FIG. 7, the display panel 11 may include a display element layer DPEL, a thin-film encapsulation layer TFEL, a touch sensing layer TSL, and an optical functional layer OFL. The display element layer DPEL, the thin-film encapsulation layer TFEL, the touch sensing layer TSL, and the optical functional layer OFL may be sequentially provided on the substrate 100.

The display element layer DPEL may include the light-emitting diode LED and a thin-film transistor TFT connected to the light-emitting diode LED. In an embodiment, FIG. 7 may schematically illustrate a portion of the pixel circuit PC described with reference to FIG. 6. For example, the thin-film transistor TFT connected to the light-emitting diode LED may correspond to a transistor, for example, the first transistor T1, connected to the light-emitting diode LED among the transistors shown in FIG. 6.

The substrate 100 may include a glass material and/or a polymer resin. In an embodiment, the substrate 100 may include a laminated structure of a base layer including a polymer resin and a barrier layer including an inorganic insulating material (e.g., an inorganic electrically insulating material). The polymer resin may include at least one selected from materials such as polyethersulfone, polyarylate, polyether imide, polyethylene naphthalate, polyethylene terephthalate, polyphenylene sulfide, polyimide, polycarbonate, cellulose triacetate, and cellulose acetate propionate. The inorganic insulating material may include at least one selected from materials such as silicon oxide (SiO₂) and silicon nitride (SiNₓ).

A first conductive layer 101 (e.g., a first electrically conductive layer 101) may be provided on the substrate 100. In an embodiment, the first conductive layer 101 may include a power line and/or a signal line. In an embodiment, the first conductive layer 101 may be provided under a semiconductor layer 103, for example, an active pattern ACT, to block or reduce transmission of radio waves and/or light, originating from a lower portion of the display panel 11, to the active pattern ACT. In an embodiment, the first conductive layer 101 may include a metal (for example, having light-shielding properties). In an embodiment, the first conductive layer 101 may include at least one selected from materials such as aluminum (Al), platinum (Pt), palladium (Pd), silver (Ag), magnesium (Mg), gold (Au), nickel (Ni), neodymium (Nd), iridium (Ir), chromium (Cr), calcium (Ca), molybdenum (Mo), titanium (Ti), tungsten (W), and copper (Cu), and may have a single-layered or multilayer structure.

A first insulating layer 102 (e.g., a first electrically insulating layer 102) may be provided on the first conductive layer 101. The first insulating layer 102 may entirely cover the first conductive layer 101. The first insulating layer 102 may have a flat top surface. The first insulating layer 102 may include an inorganic insulating material (e.g., an inorganic electrically insulating material). In an embodiment, the first insulating layer 102 may include at least one selected from materials such as silicon oxide (SiO₂), silicon nitride (SiNₓ), silicon oxynitride (SiON), aluminum oxide (Al₂O₃), titanium oxide (TiO₂), tantalum oxide (Ta₂O₅), hafnium oxide (HfO₂), and zinc oxide (ZnO₂), and may have a single-layered or multilayer structure. In an embodiment, the first insulating layer 102 may be a buffer layer.

The thin-film transistor TFT may be provided on the first insulating layer 102. The thin-film transistor TFT may include the active pattern ACT and a gate electrode GE.

The semiconductor layer 103 may be provided on the first insulating layer 102. The semiconductor layer 103 may include the active pattern ACT. The active pattern ACT may include a source area that overlaps a source electrode SE, a drain area that overlaps a drain electrode DE, and a channel area between the source area and the drain area. The channel area may overlap the gate electrode GE. The source area and the drain area may be doped with impurities (for example, dopants).

A second insulating layer 104 (e.g., a second electrically insulating layer 104) may be provided on the semiconductor layer 103. The second insulating layer 104 may cover the semiconductor layer 103, for example, the active pattern ACT. The second insulating layer 104 may include an inorganic insulating material (e.g., an inorganic electrically insulating material). In an embodiment, the second insulating layer 104 may include at least one selected from materials such as silicon oxide (SiO₂), silicon nitride (SiNₓ), silicon oxynitride (SiON), aluminum oxide (Al₂O₃), titanium oxide (TiO₂), tantalum oxide (Ta₂O₅), hafnium oxide (HfO₂), and zinc oxide (ZnO₂), and may have a single-layered or multilayer structure. In an embodiment, the second insulating layer 104 may be a first gate insulating layer.

The storage capacitor Cst may be provided on the second insulating layer 104. The storage capacitor Cst may include a first capacitor electrode CE1 and a second capacitor electrode CE2. The first capacitor electrode CE1 and the second capacitor electrode CE2 may overlap each other, and the second capacitor electrode CE2 may be provided on the first capacitor electrode CE1.

A second conductive layer 105 (e.g., a second electrically conductive layer 105) may be provided on the second insulating layer 104. The second conductive layer 105 may include the gate electrode GE of the thin-film transistor TFT and the first capacitor electrode CE1 of the storage capacitor Cst. In an embodiment, the gate electrode GE and the first capacitor electrode CE1 may be provided integrally as shown in FIG. 7. In an embodiment, the gate electrode GE and the first capacitor electrode CE1 may be provided separately. In an embodiment, the second conductive layer 105 may include at least one selected from materials such as aluminum (Al), platinum (Pt), palladium (Pd), silver (Ag), magnesium (Mg), gold (Au), nickel (Ni), neodymium (Nd), iridium (Ir), chromium (Cr), calcium (Ca), molybdenum (Mo), titanium (Ti), tungsten (W), and copper (Cu), and may have a single-layered or multilayer structure.

A third insulating layer 106 (e.g., a third electrically insulating layer 106) may be provided on the second conductive layer 105. The third insulating layer 106 may cover the second conductive layer 105, for example, the gate electrode GE and the first capacitor electrode CE1. The third insulating layer 106 may include an inorganic insulating material (e.g., an inorganic electrically insulating material). In an embodiment, the third insulating layer 106 may include at least one selected from materials such as silicon oxide (SiO₂), silicon nitride (SiNₓ), silicon oxynitride (SiON), aluminum oxide (Al₂O₃), titanium oxide (TiO₂), tantalum oxide (Ta₂O₅), hafnium oxide (HfO₂), and zinc oxide (ZnO₂), and may have a single-layered or multilayer structure. In an embodiment, the third insulating layer 106 may be a second gate insulating layer.

A third conductive layer 107 (e.g., a third electrically conductive layer 107) may be provided on the third insulating layer 106. The third conductive layer 107 may include the second capacitor electrode CE2. The second capacitor electrode CE2 may overlap the first capacitor electrode CE1. In an embodiment, the third conductive layer 107 may include at least one selected from materials such as aluminum (Al), platinum (Pt), palladium (Pd), silver (Ag), magnesium (Mg), gold (Au), nickel (Ni), neodymium (Nd), iridium (Ir), chromium (Cr), calcium (Ca), molybdenum (Mo), titanium (Ti), tungsten (W), and copper (Cu), and may have a single-layered or multilayer structure.

A fourth insulating layer 108 (e.g., a fourth electrically insulating layer 108) may be provided on the third conductive layer 107. The fourth insulating layer 108 may cover the third conductive layer 107, for example, the second capacitor electrode CE2. The fourth insulating layer 108 may include an inorganic insulating material (e.g., an inorganic electrically insulating material). In an embodiment, the fourth insulating layer 108 may include at least one selected from materials such as silicon oxide (SiO₂), silicon nitride (SiNₓ), silicon oxynitride (SiON), aluminum oxide (Al₂O₃), titanium oxide (TiO₂), tantalum oxide (Ta₂O₅), hafnium oxide (HfO₂), and zinc oxide (ZnO₂), and may have a single-layered or multilayer structure. In an embodiment, the fourth insulating layer 108 may be an interlayer insulating layer.

A fourth conductive layer 109 (e.g., a fourth electrically conductive layer 109) may be provided on the fourth insulating layer 108. The fourth conductive layer 109 may include the source electrode SE and the drain electrode DE. The source electrode SE may overlap the source area of the active pattern ACT. The source electrode SE may be connected to the active pattern ACT (for example, the source area) via openings defined in the second to fourth insulating layers 104, 106, and 108. The drain electrode DE may overlap the drain area of the active pattern ACT. The drain electrode DE may be connected to the active pattern ACT (for example, the drain area) via openings defined in the second to fourth insulating layers 104, 106, and 108. In an embodiment, the fourth conductive layer 109 may include at least one selected from materials such as aluminum (Al), platinum (Pt), palladium (Pd), silver (Ag), magnesium (Mg), gold (Au), nickel (Ni), neodymium (Nd), iridium (Ir), chromium (Cr), calcium (Ca), molybdenum (Mo), titanium (Ti), tungsten (W), and copper (Cu), and may have a single-layered or multilayer structure.

FIG. 7 shows an embodiment in which each of the second to fourth insulating layers 104, 106, and 108 has a flat top surface, but the disclosure is not necessarily limited thereto, and each of the second to fourth insulating layers 104, 106, and 108 may have a top-surface shape (for example, unevenness) corresponding to the shape of a layer provided thereunder.

A fifth insulating layer 110 (e.g., a fifth electrically insulating layer 110) may be provided on the fourth conductive layer 109. The fifth insulating layer 110 may cover the fourth conductive layer 109, for example, the source electrode SE and the drain electrode DE. The fifth insulating layer 110 may have a flat top surface. The fifth insulating layer 110 may include an organic insulating material (e.g., an organic electrically insulating material). In an embodiment, the fifth insulating layer 110 may include at least one selected from materials such as benzocyclobutene, polyimide, hexamethyldisiloxane, general-purpose polymers such as polymethylmethacrylate and/or polystyrene, polymer derivatives having phenolic groups, acrylic polymers, imide-based polymers, aryl ether-based polymers, amide-based polymers, fluorinated polymers, p-xylene-based polymers, and/or vinyl alcohol-based polymers, and may have a single-layered or multilayer structure. In an embodiment, the fifth insulating layer 110 may be the fifth insulating layer 110 may be a first via layer.

A fifth conductive layer 111 (e.g., a fifth electrically conductive layer 111) may be provided on the fifth insulating layer 110. The fifth conductive layer 111 may include a contact metal that connects the fourth conductive layer 109, for example, the drain electrode DE, with a first electrode 113 of the light-emitting diode LED. In embodiments, the fifth conductive layer 111 may also include a gate electrode of another transistor, a signal line, a power line, and/or the like. In an embodiment, the fifth conductive layer 111 may include at least one selected from materials such as aluminum (Al), platinum (Pt), palladium (Pd), silver (Ag), magnesium (Mg), gold (Au), nickel (Ni), neodymium (Nd), iridium (Ir), chromium (Cr), calcium (Ca), molybdenum (Mo), titanium (Ti), tungsten (W), and copper (Cu), and may have a single-layered or multilayer structure.

A sixth insulating layer 112 (e.g., a sixth electrically insulating layer 112) may be provided on the fifth conductive layer 111. The sixth insulating layer 112 may cover the fifth conductive layer 111. The sixth insulating layer 112 may have a flat top surface. The sixth insulating layer 112 may include an organic insulating material (e.g., an organic electrically insulating material). In an embodiment, the sixth insulating layer 112 may include at least one selected from materials such as benzocyclobutene, polyimide, hexamethyldisiloxane, general-purpose polymers such as polymethylmethacrylate and/or polystyrene, polymer derivatives having phenolic groups, acrylic polymers, imide-based polymers, aryl ether-based polymers, amide-based polymers, fluorinated polymers, p-xylene-based polymers, and/or vinyl alcohol-based polymers, and may have a single-layered or multilayer structure. In an embodiment, the sixth insulating layer 112 may be a second via layer.

The light-emitting diode LED may be provided on the sixth insulating layer 112. The light-emitting diode LED may include the first electrode 113 and a second electrode 119, facing each other, and an intermediate layer 115 between the first electrode 113 and the second electrode 119. In an embodiment, the first electrode 113 of the light-emitting diode LED may correspond to an anode, and the second electrode 119 may correspond to a cathode.

The first electrode 113 of the light-emitting diode LED may be provided on the sixth insulating layer 112. The first electrode 113 may be connected to the fifth conductive layer 111 via an opening defined in the sixth insulating layer 112. The first electrode 113 may be (for example, electrically) connected to the thin-film transistor TFT via the fifth conductive layer 111 and the drain electrode DE. The first electrode 113 may include a conductive oxide (e.g., an electrically conductive oxide) such as indium tin oxide (ITO), indium zinc oxide (IZO), zinc oxide (ZnO), indium oxide (In₂O₃), indium gallium oxide (IGO), and/or aluminum zinc oxide (AZO). The first electrode 113 may include a reflective film including silver (Ag), magnesium (Mg), aluminum (Al), platinum (Pt), palladium (Pd), gold (Au), nickel (Ni), neodymium (Nd), iridium (Ir), chromium (Cr), and/or a compound thereof. However, the configuration and material of the first electrode 113 are not limited thereto and may be changed in various suitable ways.

A pixel-defining layer 114 may be provided on the sixth insulating layer 112. The pixel-defining layer 114 may cover an edge of the first electrode 113. For example, the pixel-defining layer 114 may be opened to expose a central portion of the first electrode 113. The size and shape of an emission area of the light-emitting diode LED may be determined by an opening in the pixel-defining layer 114. In an embodiment, the pixel-defining layer 114 may include an organic insulating material (e.g., an organic electrically insulating material). In an embodiment, the pixel-defining layer 114 may include at least one of materials such as benzocyclobutene, polyimide, hexamethyldisiloxane, general-purpose polymers such as polymethylmethacrylate and/or polystyrene, polymer derivatives having phenolic groups, acrylic polymers, imide-based polymers, aryl ether-based polymers, amide-based polymers, fluorinated polymers, p-xylene-based polymers, and/or vinyl alcohol-based polymers, and may have a single-layered or multilayer structure.

The intermediate layer 115 may be provided on the first electrode 113. The intermediate layer 115 may include first and second common layers 116 and 118 provided on the pixel-defining layer 114, and an emission layer 117 provided in the opening in the pixel-defining layer 114. The first common layer 116 may be provided on the second common layer 118. In an embodiment, the first common layer 116 may be provided on the pixel-defining layer 114, the emission layer 117 may be provided on the first common layer 116 and in the opening in the pixel-defining layer 114, and the second common layer 118 may be provided on the first common layer 116 to cover the emission layer 117. For example, the emission layer 117 may be provided in the opening in the pixel-defining layer 114 and may be provided between the first common layer 116 and the second common layer 118.

The emission layer 117 may include a low-molecular-weight material and/or polymer material that emits light of a set or specific color if (e.g., when) a current flows. The first common layer 116 may include an electron transport layer (ETL) and/or an electron injection layer (EIL). The second common layer 118 may include a hole transport layer (HTL) and/or a hole injection layer (HIL). In some embodiments, the first common layer 116 and/or the second common layer 118 may be omitted. In some embodiments, the positions of the first common layer 116 and the second common layer 118 may be interchanged with each other.

The second electrode 119 may be provided on the intermediate layer 115. For example, the second electrode 119 may be provided on the second common layer 118. The second electrode 119 may be provided to entirely cover the intermediate layer 115. The second electrode 119 may include a conductive material (e.g., an electrically conductive material). For example, the second electrode 119 may include a transparent layer (or a semitransparent layer) including silver (Ag), magnesium (Mg), aluminum (Al), platinum (Pt), palladium (Pd), gold (Au), nickel (Ni), neodymium (Nd), iridium (Ir), chromium (Cr), lithium (Li), calcium (Ca), and/or alloys thereof. In embodiments, the second electrode 119 may further include a layer including an oxide such as indium tin oxide (ITO), indium zinc oxide (IZO), zinc oxide (ZnO), and/or indium oxide (In₂O₃) on the transparent layer (or the semi-transparent layer) including the above-described material.

The thin-film encapsulation layer TFEL may be provided on the second electrode 119 and may entirely cover the light-emitting diode LED. The thin-film encapsulation layer TFEL may include at least one inorganic layer and at least one organic layer. In an embodiment, the thin-film encapsulation layer TFEL may include a first inorganic encapsulation layer 120, a second inorganic encapsulation layer 122, and an organic encapsulation layer 121 provided between the first inorganic encapsulation layer 120 and the second inorganic encapsulation layer 122. In an embodiment, each of the first inorganic encapsulation layer 120 and the second inorganic encapsulation layer 122 may have a top-surface shape (for example, unevenness) according to the shape of a layer provided thereunder. In an embodiment, the organic encapsulation layer 121 may have a flat top surface.

The first inorganic encapsulation layer 120 and/or the second inorganic encapsulation layer 122 may include at least one selected from materials such as silicon oxide (SiO₂), silicon nitride (SiNₓ), silicon oxynitride (SiON), aluminum oxide (Al₂O₃), titanium oxide (TiO₂), tantalum oxide (Ta₂O₅), hafnium oxide (HfO₂), and zinc oxide (ZnO₂), and may have a single-layered or multilayer structure. The organic encapsulation layer 121 may include a polymer-based material. The polymer-based material may include, for example, silicone-based resin, acrylic resin, epoxy-based resin, polyimide, and/or polyethylene.

The touch sensing layer TSL may be provided on the thin-film encapsulation layer TFEL. The touch sensing layer TSL may include touch electrode layers to detect external inputs, for example, touch events, and a touch insulating layer (e.g., a touch electrically insulating layer) between the touch electrode layers. The touch sensing layer TSL may include a first touch insulating layer 123 (e.g., a first touch electrically insulating layer 123), a first touch electrode layer 124, a second touch insulating layer 125 (e.g., a second tough electrically insulating layer 125), a second touch electrode layer 126, and a third touch insulating layer 127 (e.g., a third touch electrically insulating layer 127).

The first touch insulating layer 123 may be provided on the thin-film encapsulation layer TFEL, for example, the second inorganic encapsulation layer 122 of the thin-film encapsulation layer TFEL. The first touch electrode layer 124 may be provided on the first touch insulating layer 123. The second touch insulating layer 125 may be provided on the first touch electrode layer 124 and may cover the first touch electrode layer 124. The second touch electrode layer 126 may be provided on the second touch insulating layer 125. The third touch insulating layer 127 may be provided on the second touch electrode layer 126 and may cover the second touch electrode layer 126. The second touch electrode layer 126 may be connected to the first touch electrode layer 124 via an opening defined in the second touch insulating layer 125. In an embodiment, the second touch electrode layer 126 may include a sensing electrode, and the first touch electrode layer 124 may include a bridge electrode.

In an embodiment, the first touch insulating layer 123 may include an inorganic insulating material (e.g., an inorganic electrically insulating material). In an embodiment, the first touch insulating layer 123 may include at least one selected from materials such as silicon oxide (SiO₂), silicon nitride (SiNₓ), silicon oxynitride (SiON), aluminum oxide (Al₂O₃), titanium oxide (TiO₂), tantalum oxide (Ta₂O₅), hafnium oxide (HfO₂), and zinc oxide (ZnO₂), and may have a single-layered or multilayer structure.

In an embodiment, each of the second touch insulating layer 125 and the third touch insulating layer 127 may include an organic insulating material (e.g., an organic electrically insulating material). In an embodiment, the second touch insulating layer 125 and/or the third touch insulating layer 127 may include an acrylic organic insulating material (e.g., an acrylic organic electrically insulating material). In an embodiment, the second touch insulating layer 125 and/or the third touch insulating layer 127 may have a flat top surface.

The first touch electrode layer 124 and the second touch electrode layer 126 may not entirely overlap the emission area of the light-emitting diode LED. In an embodiment, the first touch electrode layer 124 and the second touch electrode layer 126 may not overlap the emission layer 117 of the light-emitting diode LED. In other words, each of the first touch electrode layer 124 and the second touch electrode layer 126 may have an opening that overlaps the emission area of the light-emitting diode LED or the emission layer 117.

The optical functional layer OFL may be provided on the touch sensing layer TSL. The optical functional layer OFL may include a light-shielding layer 128, a color filter 129, and an overcoat layer 130. The overcoat layer 130 may cover the light-shielding layer 128 and the color filter 129 and may have a flat top surface.

The light-shielding layer 128 may be provided ed on the touch sensing layer TSL, for example, the third touch insulating layer 127. The light-shielding layer 128 may include a light-shielding material, and by covering the first and second touch electrode layers 124 and 126, may prevent or reduce reflection of external light off the first and second touch electrode layers 124 and 126 that would otherwise make the first and second touch electrode layers 124 and 126 visible to a user. However, the light-shielding layer 128 is not limited to this effect and may provide various other suitable effects. In embodiments, the light-shielding layer 128 may be opened to allow transmission of light emitted from the light-emitting diode LED. An opening in the light-shielding layer 128 may overlap the light-emitting diode LED, for example, the emission layer 117.

The color filter 129 may be provided on the light-shielding layer 128. The color filter 129 may overlap the opening in the light-shielding layer 128, and a portion of the color filter 129 may be provided in the opening in the light-shielding layer 128. The color filter 129 may allow transmission of light of a color (for example, a wavelength band) corresponding to light emitted from the emission layer 117 of the light-emitting diode LED. For example, if (e.g., when) the emission layer 117 of the light-emitting diode LED emits red light, the color filter 129 may be configured to transmit light in a red wavelength band.

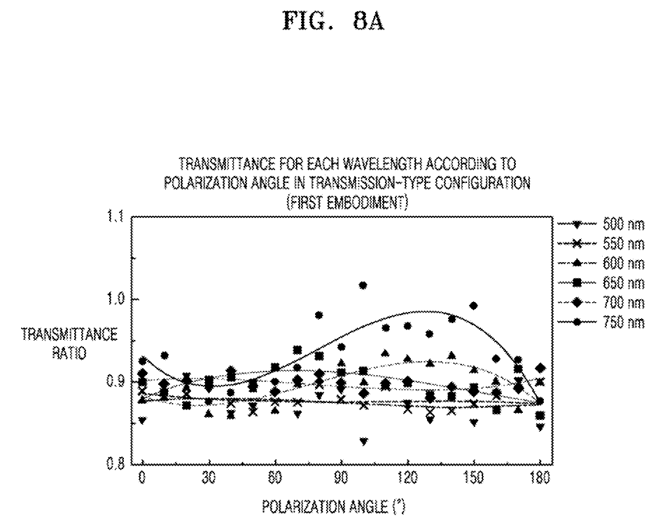

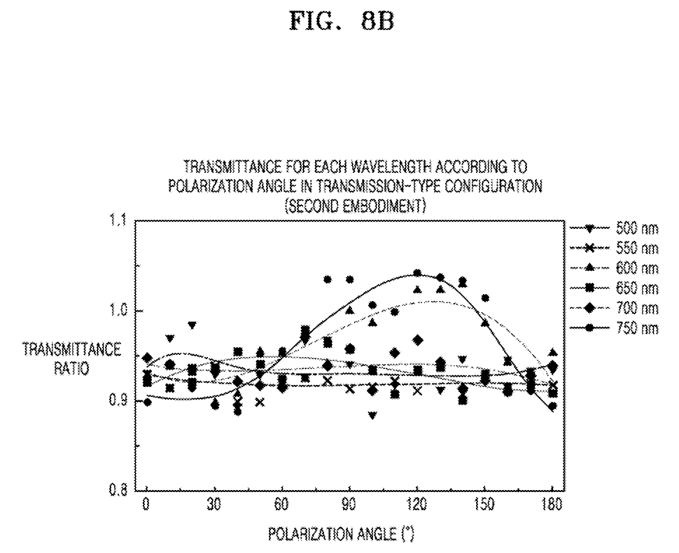

FIG. 8A is a graph of a transmittance ratio with respect to a polarization angle according to an embodiment. FIG. 8B is a graph of a transmittance ratio with respect to a polarization angle according to an embodiment.

Referring to FIG. 8A, a graph is shown illustrating a transmittance ratio for each wavelength according to a polarization angle if (e.g., when) the transmission-type configuration shown in FIG. 1 is combined with the polarization structure in the first embodiment shown in FIGS. 3A to 3C.

Referring to FIG. 8B, a graph is shown illustrating a transmittance ratio for each wavelength according to a polarization angle if (e.g., when) the transmission-type configuration shown in FIG. 1 is combined with the polarization structure in the second embodiment shown in FIGS. 4A to 4C.

In the graphs of FIGS. 8A and 8B, the x-axis represents the polarization angle of a polarizer, with units in degrees (°). The y-axis represents the transmittance ratio, which is the ratio of the transmittance of a polarizer with the polarization structure applied relative to the transmittance of, for example, a film without the polarization structure.

In an embodiment, referring to FIG. 8A, for a wavelength of 750 nanometers (nm), the transmittance ratio is about 0.93 at a polarization angle of 0 degrees, and the transmittance ratio is about 0.95 at a polarization angle of 90 degrees. In an embodiment, referring to FIG. 8B, for a wavelength of 750 nanometers (nm), the transmittance ratio is about 0.90 at a polarization angle of 0 degrees, and the transmittance ratio is about 1.03 at a polarization angle of 90 degrees. Therefore, a difference in transmittance ratio corresponding to a 90-degree difference in polarization angle for light of a wavelength of 750 nanometers (nm) may be about 0.02 in the embodiment shown in FIG. 8A and about 0.13 in the embodiment shown in FIG. 8B.

In an embodiment, referring to FIG. 8A, for a wavelength of 750 nanometers (nm), the transmittance ratio is about 0.89 at a polarization angle of 30 degrees, and the transmittance ratio is about 0.98 at a polarization angle of 120 degrees. In an embodiment, referring to FIG. 8B, for a wavelength of 750 nanometers (nm), the transmittance ratio is about 0.90 at a polarization angle of 30 degrees, and the transmittance ratio is about 1.04 at a polarization angle of 120 degrees. Therefore, a difference in transmittance ratio corresponding to a 90-degree difference in polarization angle for light of a wavelength of 750 nanometers (nm) may be about 0.09 in the embodiment shown in FIG. 8A and about 0.14 in the embodiment shown in FIG. 8B.

In an embodiment, referring to FIG. 8A, for a wavelength of 750 nanometers (nm), the minimum transmittance ratio is about 0.89, and the maximum transmittance ratio is about 0.98. Referring to FIG. 8B, for a wavelength of 750 nanometers (nm), the minimum transmittance ratio is about 0.90, and the maximum transmittance ratio is about 1.04. Therefore, a difference between the maximum and minimum transmittance ratios for light of a wavelength of 750 nanometers (nm) may be about 0.09 in the embodiment shown in FIG. 8A and about 0.14 in the embodiment shown in FIG. 8B.

Comparing FIG. 8A with FIG. 8B, it may be confirmed that in the transmission-type configuration, the transmittance varies more dramatically depending on the polarization angle if (e.g., when) the polarization structure in the second embodiment is utilized. A difference between the first embodiment and the second embodiment lies in the length of the filaments in the connection structure of microorganisms of the polarization structure, and thus, it may be confirmed that the amount of transmittance (e.g., light transmittance) variation with respect to changes in polarization angle may be adjusted by adjusting the length of the filaments of the microorganisms.

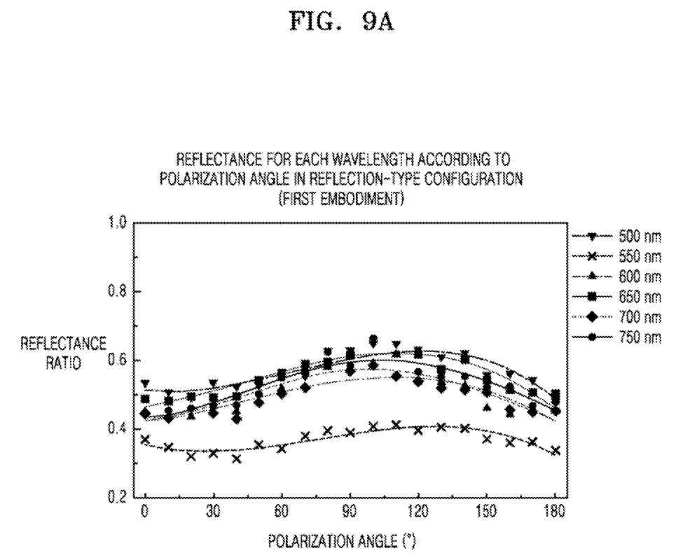

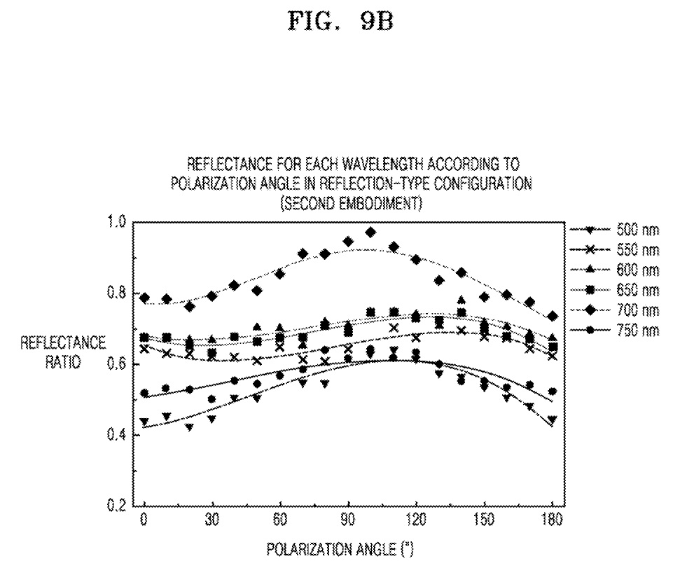

FIG. 9A is a graph of a reflectance ratio with respect to a polarization angle according to an embodiment. FIG. 9B is a graph of a reflectance ratio with respect to a polarization angle according to an embodiment.

Referring to FIG. 9A, a graph is shown illustrating a reflectance ratio for each wavelength according to a polarization angle if (e.g., when) the reflection-type configuration shown in FIG. 2 is combined with the polarization structure in the first embodiment shown in FIGS. 3A to 3C.

Referring to FIG. 9B, a graph is shown illustrating a reflectance ratio for each wavelength according to a polarization angle if (e.g., when) the transmission-type configuration shown in FIG. 2 is combined with the polarization structure in the second embodiment shown in FIGS. 4A to 4C.

In the graphs of FIGS. 9A and 9B, the x-axis represents the polarization angle of a polarizer, with units in degrees (°). The y-axis represents the reflectance ratio, which is the ratio of the reflectance of a polarizer with the polarization structure applied relative to the reflectance of, for example, a mirror without the polarization structure.

In an embodiment, referring to FIG. 9A, for a wavelength of 500 nanometers (nm), the reflectance ratio may be about 0.50 to about 0.62. In an embodiment, referring to FIG. 9B, for a wavelength of 500 nanometers (nm), the reflectance ratio may be about 0.40 to about 0.60.

In an embodiment, referring to FIG. 9A, for a wavelength of 550 nanometers (nm), the reflectance ratio may be about 0.30 to about 0.38. In an embodiment, referring to FIG. 9B, for a wavelength of 550 nanometers (nm), the reflectance ratio may be about 0.60 to about 0.66.

In an embodiment, referring to FIG. 9A, for a wavelength of 600 nanometers (nm), the reflectance ratio may be about 0.44 to about 0.62. In an embodiment, referring to FIG. 9B, for a wavelength of 600 nanometers (nm), the reflectance ratio may be about 0.67 to about 0.73.

In an embodiment, referring to FIG. 9A, for a wavelength of 650 nanometers (nm), the reflectance ratio may be about 0.46 to about 0.60. In an embodiment, referring to FIG. 9B, for a wavelength of 650 nanometers (nm), the reflectance ratio may be about 0.64 to about 0.74.

In an embodiment, referring to FIG. 9A, for a wavelength of 700 nanometers (nm), the reflectance ratio may be about 0.44 to about 0.58. In an embodiment, referring to FIG. 9B, for a wavelength of 700 nanometers (nm), the reflectance ratio may be about 0.74 to about 0.96.

In an embodiment, referring to FIG. 9A, for a wavelength of 750 nanometers (nm), the reflectance ratio may be about 0.44 to about 0.62. In an embodiment, referring to FIG. 9B, for a wavelength of 750 nanometers (nm), the reflectance ratio may be about 0.51 to about 0.60.

Comparing FIG. 9A with 9B, it may be confirmed that in the reflection-type configuration, the second embodiment exhibits a greater variation in reflectance with respect to wavelength than the first embodiment. Therefore, a range in which reflectance varies according to wavelength may be adjusted by adjusting the length of the filaments of the microorganisms included in the polarization structure.

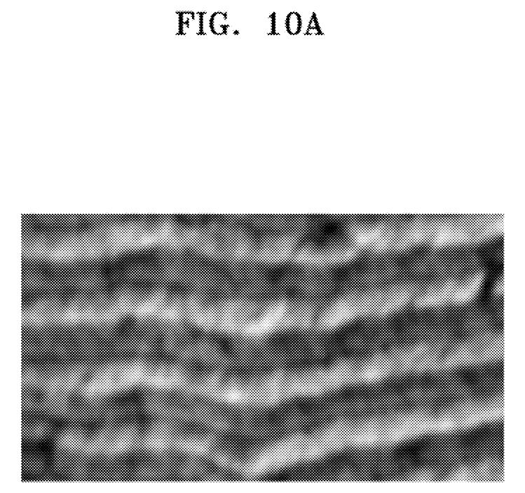

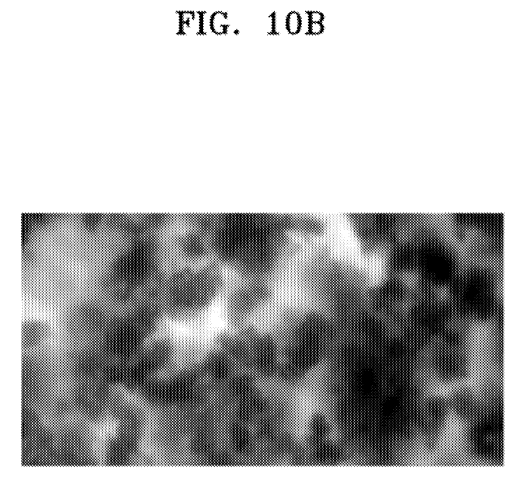

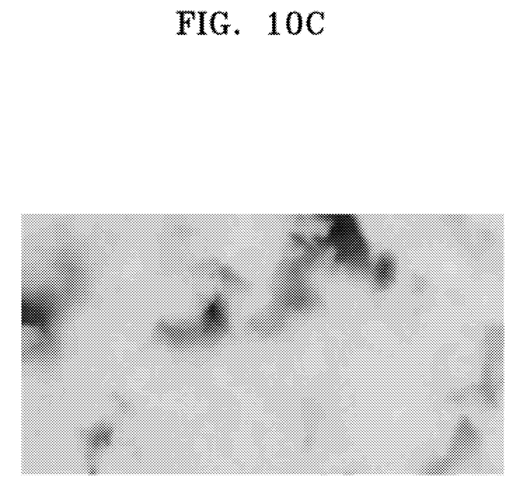

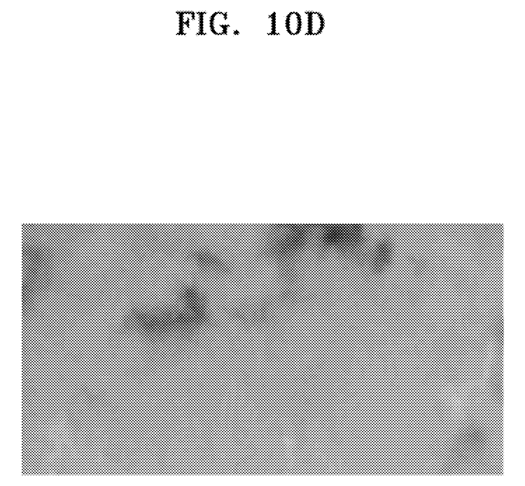

FIGS. 10A, 10B, 10C, and 10D are images of set or certain areas of an object for inspection.

FIG. 10A may be an image obtained based on light (for example, the second light L2 in FIG. 1) that has not passed through a polarizer. FIG. 10B may be an image obtained based on light (for example, the third light L3) that has passed through the first polarizer 221 in FIG. 1. FIG. 10C may be an image obtained based on light (for example, the fourth light L4) that has passed through the second polarizer 222 in FIG. 1. FIG. 10D may be an image obtained based on light (for example, the fifth light L5) that has passed through the third polarizer 223 in FIG. 1.

Referring to FIGS. 10A to 10D, it may be confirmed that the number of components shown in the image decreases as the light passes through the polarizer(s). The image of FIG. 10D, obtained after the light passes through all of the first to third polarizers 221, 222, and 223, may be used to identify characteristics of an object for inspection.

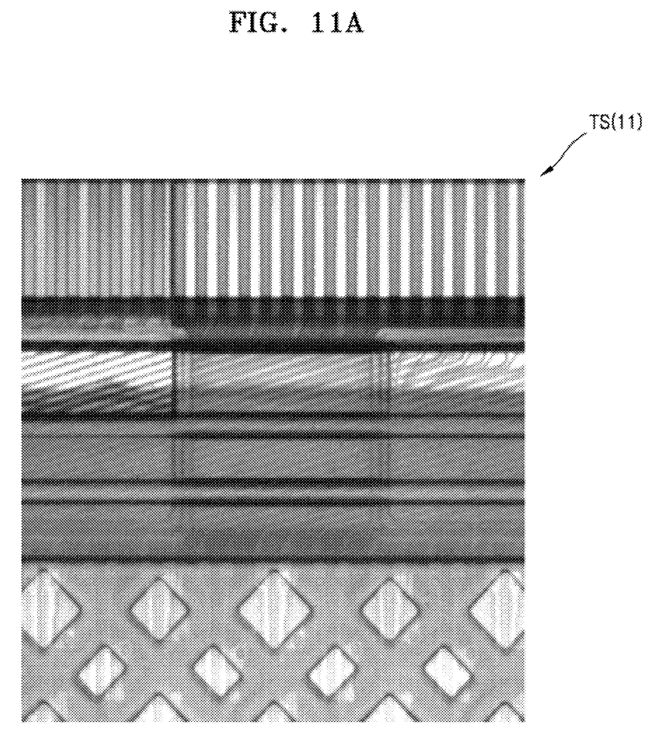

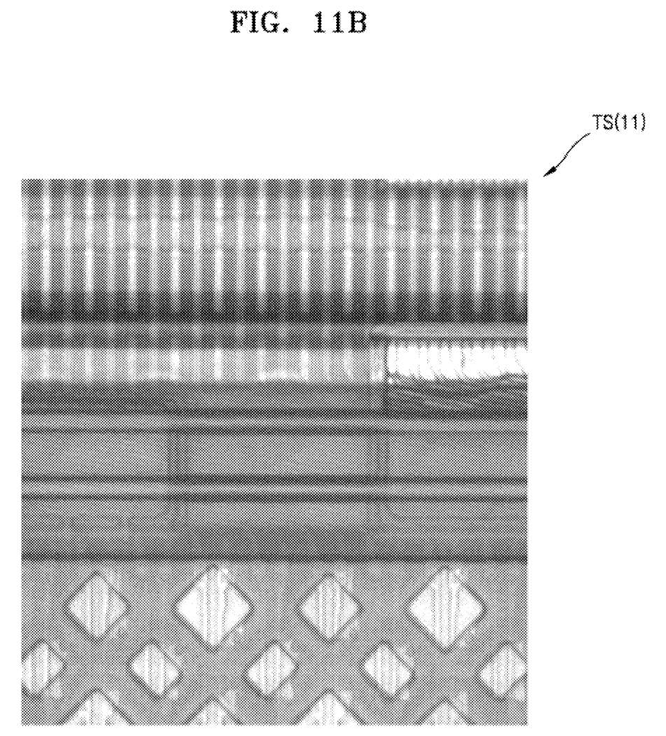

FIG. 11A is a plan view of a portion of a display panel according to an embodiment. FIG. 11B is a plan view of a portion of a display panel according to an embodiment.

FIGS. 11A and 11B show the display panel 11 as the object TS for inspection. FIG. 11A shows an embodiment in which a set or specific layer, for example, the organic encapsulation layer 121 (see FIG. 7), of the display panel 11 is not provided, and FIG. 11B shows an embodiment in which the set or specific layer, for example, the organic encapsulation layer 121 (see FIG. 7), of the display panel 11 is provided.

FIGS. 11A and 11B may be images generated based on light reflected from the object TS for inspection (for example, the display panel 11) and detected by the sensor 23 after passing through the at least one polarizer 22. In an embodiment, the processor 24 may be configured to form an image having a higher gray level, for example, a more blurred image, after analyzing the light detected by the sensor 23, if (e.g., when) the object TS for inspection (for example, the display panel 11) includes a material of the organic encapsulation layer 121, for example, a polymer-based organic insulating material (e.g., a polymer-based organic electrically insulating material). Accordingly, the image of FIG. 11B is shown as a blurry image having a generally higher gray level than the image of FIG. 11A.

According to an embodiment as described above, an optical inspection device including a polarizer having a polarization structure formed by a connection structure of microorganisms may be provided. Because the connection structure formed by self-replication of the microorganisms is obtained and used as the polarization structure, the nanometer-scale polarization structure utilized or required for the polarizer may be easily formed. In embodiments, to implement various suitable polarization structures of the polarizer, simple variables such as the degree of self-replication of the microorganisms, for example, the replication time, may be adjusted, thereby making control of formation of the polarization structure easier. The disclosure provides embodiments of an optical inspection device including a polarizer having a polarization structure, an optical inspection system including the optical inspection device, and an optical inspection method using the optical inspection system.

It should be understood that embodiments described herein should be considered in a descriptive sense only and not for purposes of limitation. Descriptions of features or aspects within each embodiment should generally be considered as available for other similar features or aspects in other embodiments. While one or more embodiments have been described with reference to the figures, it will be understood by those of ordinary skill in the art that various suitable changes in form and details may be made therein without departing from the spirit and scope as defined by the following claims, and equivalents thereof.

What is claimed is:

1. An optical inspection device comprising:
a light source;
at least one polarizer provided on a path of light emitted from the light source and comprising a polarization structure; and
a sensor that detects light from the at least one polarizer,
wherein the polarization structure of the at least one polarizer comprises a connection structure that comprises a plurality of microorganisms.

2. The optical inspection device of claim 1, wherein the connection structure that comprises the plurality of microorganisms is provided as a mesh structure that comprises holes that generally extend in one direction.

3. The optical inspection device of claim 1, wherein the plurality of microorganisms comprise filamentous bacteriophages.

4. The optical inspection device of claim 1, wherein the at least one polarizer comprises a film that transmits the light emitted from the light source.

5. The optical inspection device of claim 1, wherein the at least one polarizer comprises a mirror that reflects the light emitted from the light source.

6. The optical inspection device of claim 1, further comprising a processor connected to the sensor and comprising an algorithm that creates an image based on the light detected by the sensor.

7. The optical inspection device of claim 1, wherein the at least one polarizer comprises a protective film that covers the polarization structure.

8. The optical inspection device of claim 1, wherein the at least one polarizer comprises a plurality of polarizers having different polarization angles and provided continuously on the path of the light emitted from the light source.

9. An optical inspection system comprising:
a light source;
an object for inspection provided on a path of light emitted from the light source;
at least one polarizer provided on the path of the light emitted from the light source and reflected from the object for inspection, and comprising a polarization structure; and
a sensor that detects light from the at least one polarizer,
wherein the polarization structure of the at least one polarizer comprises a connection structure that comprises a plurality of microorganisms.

10. The optical inspection system of claim 9, wherein the connection structure that comprises the plurality of microorganisms is provided as a mesh structure that comprises holes that generally extend in one direction.

11. An optical inspection method comprising:
irradiating light from a light source toward an object for inspection;
polarizing, at least once, light reflected from the object for inspection, using at least one polarizer; and
detecting, using a sensor, the light polarized by the at least one polarizer,
wherein a polarization structure of the at least one polarizer comprises a connection structure that comprises a plurality of microorganisms.

12. The optical inspection method of claim 11, wherein the connection structure that comprises the plurality of microorganisms is provided as a mesh structure that comprises holes that generally extend in one direction.

13. The optical inspection method of claim 11, wherein the plurality of microorganisms comprise filamentous bacteriophages.

14. The optical inspection method of claim 11, wherein the at least one polarizer comprises a film that transmits light, and
in the polarizing of the light, the light is polarized by the polarization structure and is transmitted through the film.

15. The optical inspection method of claim 11, wherein the at least one polarizer comprises a mirror that reflects light, and
in the polarizing of the light, the light is polarized by the polarization structure and is reflected by the mirror.

16. The optical inspection method of claim 11, further comprising forming an image of the object for inspection, based on the light detected by the sensor.

17. The optical inspection method of claim 16, wherein the at least one polarizer comprises a protective film that covers the polarization structure, and
the forming of the image comprises compensating for an effect of the protective film on the light.

18. The optical inspection method of claim 11, wherein, in the polarizing of the light, the light reflected from the object for inspection is polarized a plurality of times using a plurality of polarizers provided continuously on a path of light and having different polarization angles.

19. The optical inspection method of claim 11, wherein the object for inspection comprises a display panel, and characteristics of a set area of the display panel are identified based on the light detected using the sensor.

20. The optical inspection method of claim 19, wherein the object for inspection is moved to identify whether a set layer of the display panel is present and to identify a boundary of the set layer.
